(19) United States
(12) Patent Application Publication
(10) Pub. No.: US 2024/0408270 A1
(43) Pub. Date: Dec. 12, 2024

(54) IMPLANT IMAGING SYSTEM

(71) Applicant: MERIL HEALTHCARE PVT. LTD, Vapi (IN)

(72) Inventors: PRAMOD KUMAR MINOCHA, Vapi (IN); DEVESHKUMAR MAHENDRALAL KOTHWALA, Surat (IN); ARPIT PRADIPKUMAR DAVE, Silvassa (IN)

(73) Assignee: MERIL HEALTHCARE PVT. LTD, Vapi (IN)

(21) Appl. No.: 18/697,648

(22) PCT Filed: Feb. 24, 2022
(86) PCT No.: PCT/IN2022/050160

§ 371 Date: Apr. 1, 2024

(30) Foreign Application Priority Data
Oct. 18, 2021 (IN) 202121047197

(51) Int. Cl. A61L 27/18; A61F 2/30; A61F 2/34; A61F 2/38; A61L 27/32; A61L 27/54

(52) U.S. Cl. CPC A61L 27/18; A61F 2/30767; A61F 2/34; A61F 2/3859; A61L 27/32; A61L 27/54; A61F 2002/3008; A61F 2002/3093; A61F 2310/00796; A61L 2430/24

(57) ABSTRACT

An implant imaging system (200) is disclosed. The implant imaging system 200 may be provided on a joint implant made of polymer. The implant imaging system (200) includes one or more metal plates (212) including at least one bone contacting surface ‘B’. The metal plates (212) are disposed on a joint implant. Further, the bone contacting surface ‘B’ metal plates (212) are coated with one or more coatings of osteoconductive material. The presence of osteoconductive material increases the rate of osteointegration of the joint implant.

### FIELD OF INVENTION

The present invention relates to an implant imaging system. More specifically, the present invention relates to the implant imaging system for joint implants.

### BACKGROUND

There are different types of joints in a human body for example a knee joint, hip joint, elbow joint, shoulder joint to name a few. Basically, a joint is formed where ends of two or more bones meet. Healthy joints are important to perform everyday activities.

Several conditions can cause joint pain and disability. For example, the joint pain and disability may be caused by damage to the articular cartilage, a smooth substance that protects the bones and enables them to move easily within the joint. Normally all components of the joint work in harmony. But disease or injury can disrupt this harmony, resulting in pain, muscle weakness, and reduced function. For example, in the case of a knee joint, the most common cause of chronic joint pain and/or disability is arthritis.

If nonsurgical treatments like medications, physical therapy, and activity modifications do not relieve the pain and disability, the doctor may recommend joint replacement surgery.

The implants used in the joint replacement surgery may include unique systems having been designed to replace damaged, diseased, or dysfunctional native structures. Conventionally, the implants use a combination of cobalt chromium (CoCrMo) alloy and ultra-high molecular weight polyethylene (UHMWPE). For example, in knee implant, the femoral condyle and tibial tray (tibia platform) are made of CoCrMo alloy and the rotatable or slidable tibial pad is made of ultra-high molecular weight polyethylene.

However, the conventional metal materials, like CoCrMo alloys, have several clinical drawbacks while being used as femoral condyles or tibial trays. Further, joint replacement implants are subjected to a high degree of load and wear inducing movement that results in the release of abrasive particles. Contact stress between the implant's polyethylene bearing surfaces and the metal components can cause wear leading to release of metal ions. Wear debris and metal ions are the main cause of inflammation, premature loosening and metal toxicity associated with joint replacement implants. Further, CoCrMo alloys contains a small amount of nickel which can cause allergic reactions inside the human body.

Moreover, the conventional implants are not compatible with known diagnostic device like computed tomography (CT) and magnetic resonance imaging (MRI) techniques, due to which physicians are not able to track the bone growth and healing post-implantation.

Due to these drawbacks, a new generation of polymer material poly-ether ether ketone (PEEK) with high chemical stability, high strength and high biocompatibility are widely used in orthopedic endophytes.

However, Polyether ether ketone (PEEK) material is not visible (poor radio opacity) in an X-ray. Even if the Polyether ether ketone (PEEK) substrates are provided with a titanium coating or a hydroxyapatite layer, the implants remain largely invisible in an X-ray image. Conventionally, in order to solve the problem of low radiopacity, metal inlays have been used in the joint implant. One such implant with the metal inlays is disclosed in application number ES2815658T3. However, such metal inlays do not completely address the problem of low radiopacity in an effective manner. The metal inlays of the said application have limited visibility and fails to display whole structure of the implant for precise placement as well as post-surgery follow-ups.

Further for implantation purposes, it is very important to have cell adhesion property which is governed by surface chemistry and topography of the implant. But Polyether ether ketone (PEEK) material has low osteointegration due to its high chemical stability. Hence its inferior bioactivity may lead to low bone implant interaction.

Therefore, there arises a requirement of an imaging system which can overcome existing problems of the joint implants.

### SUMMARY

The present invention relates to an implant imaging system. The implant imaging system may be provided on a joint implant made of polymer. The implant imaging system 200 includes one or more metal plates including at least one bone contacting surface ‘B’. The metal plates are disposed on a joint implant. Further, the bone contacting surface ‘B’ metal plates are coated with one or more coatings of osteoconductive material. The presence of osteoconductive material increases the rate of osteointegration of the joint implant.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an implant imaging system 200 in accordance with an embodiment of the present invention.

FIG. 1a depicts an implant 100 in accordance with an embodiment of the present invention.

FIG. 2a-b depicts a femoral component 110 in accordance with an embodiment of the present invention.

FIG. 3a-b depicts a femoral component 210 in accordance with an embodiment of the present invention.

FIG. 4a-f depicts a femoral component 220 in accordance with an embodiment of the present invention.

FIG. 5a-f depicts a femoral component 230 in accordance with an embodiment of the present invention.

FIG. 6a-b depicts markers 171, 173 in accordance with an embodiment of the present invention.

FIG. 7 depicts a hip implant 300 in accordance with an embodiment of the present invention.

FIG. 8a depicts an X-ray image of a conventional implant in accordance with an embodiment of the present invention.

FIG. 8b depicts an X-ray image of the femoral component 230 in accordance with an embodiment of the present invention.

### DETAILED DESCRIPTION OF THE DRAWINGS

Prior to describing the invention in detail, definitions of certain words or phrases used throughout this patent document will be defined: the terms “include” and “comprise”, as well as derivatives, mean inclusion without limitation; the term “or” is inclusive, meaning and/or; Definitions of certain words and phrases are provided throughout this patent document, and those of ordinary skill in the art will understand that such definitions apply in many, if not most, instances to prior as well as future uses of such defined words and phrases.

Wherever possible, same reference numbers will be used throughout the drawings to refer to same or like parts. Moreover, references to various elements described herein are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

Particular embodiments of the present disclosure are described herein below with reference to the accompanying drawings, however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In accordance with the present disclosure, an implant imaging system is disclosed. The implant imaging system of the present invention imparts enhanced visibility through radiological instruments and/or promotes osteointegration.

In an embodiment, the implant imaging system of the present invention includes one or more metal plates having a bone contacting surface. In an embodiment, the bone contacting surface of the metal plate is coated with an osteoconductive material. The metal plate improves the radiopacity of the implant leading to better visualization during the surgery, thus, enabling precise implantation and better post-surgery follow ups through radiological instruments. The metal component provides higher visibility in radiopaque techniques therefore, the implant can be easily used with complex geometry bone structure.

The coating of an osteoconductive material on the metal plate helps in better osteointegration and provides long term stability of the implant. Further, the coating on the metal component facilitates strong adhesion to the bone, minimizes inflammation, prevent premature loosening, metal toxicity and allergic reaction within the body.

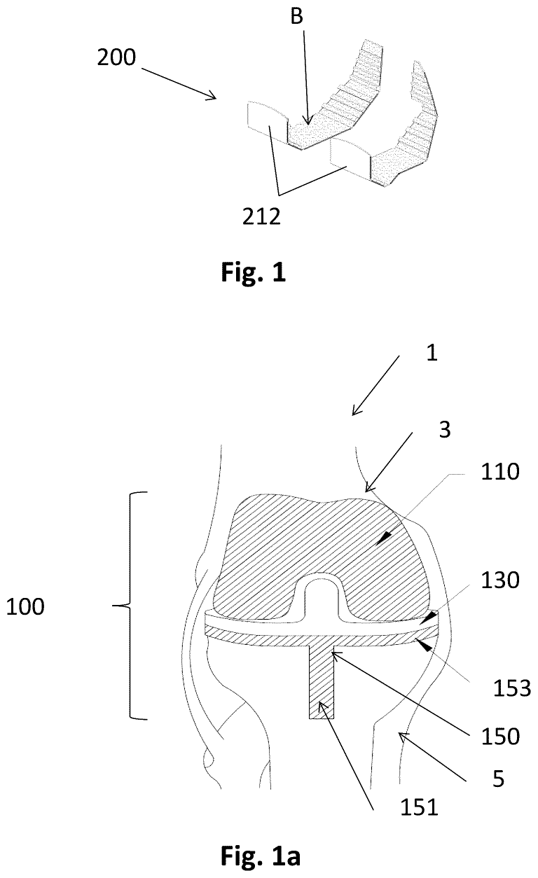

Now referring specifically to drawings, FIG. 1 represents an implant imaging system 200. The implant imaging system 200 may include one or more components such as but not limited to one more metal plates 212 having a bone contacting surface ‘B’ and one or more layers of coatings on the bone contacting surface ‘B’ of the metal plates 212.

The implant imaging system 200 may be provided on a joint implant, for example, a knee implant 100 (depicted in FIG. 1A) at a predefined position. The predefined position may be a portion of the joint implant which remains in contact with the native bone structure in order to be visualized under radiographic examinations and/or where maximum in-bone growth is required. The joint implant may be made of a polymeric material. The metal plates 212 of the implant imaging system 200 helps in adequate visibility for precise placement of the joint implant during surgery and post-surgery follow-ups.

The metal plate 212 may be made of metallic material but not limited to titanium, tantalum, gold, platinum, hafnium, CoCrMo alloy or a combination (alloy) thereof. In an embodiment, the metal plate 212 is made of CoCrMo alloy.

The implant imaging system 200 may be provided on a joint implant, The joint implant may include a spine implant, a shoulder implant, a hip implant 300 or a knee implant 100 (depicted in FIG. 1A) at a predefined position. The predefined position may be a portion of the joint implant which remains in contact with the native bone structure in order to be visualized under radiographic examinations and/or where maximum in-bone growth is required. The metal plates 212 of the implant imaging system 200 may be disposed on the joint implant. The metal plates 212 of the implant imaging system 200 helps in adequate visibility for precise placement of the joint implant during surgery and post-surgery follow-ups. The metal plate 212 may be attached to the joint implant by means of without limitation, snap fit, press fil, adhesives etc. In an embodiment, the metal plate 212 is disposed by means of the snap fit mechanism.

The metal plate 212 may include a predefined thickness ranging from 1 mm to 3 mm, preferably 1.5 mm to 2 mm. In an embodiment, the thickness of the metal plate 212 is 1.5 mm. In various embodiments, the metal plate 212 may be a singular structure and/or multiple structures depending upon the shape and dimensions of the joint implant as depicted in FIGS. 3-5.

The bone contacting surface ‘B’ of the metal plate 212 may be defined as the side which remains in contact with the natural bone upon implantation of the joint implant. The surface ‘B’ may be coated with one or more coatings of osteoconductive material. The osteoconductive material may include without limitation Calcium Sulfate, bioactive glass ceramics, Hydroxyapatite, tricalcium phosphate or combination thereof. In an embodiment, the surface ‘B’ is coated with the hydroxyapatite (HA).

The HA coating may have an optimum thickness such that it does not produce any defect which may affect rate of osseointegration. The thickness may be in a range of 110 μm to 190 μm. In an embodiment, the thickness of the HA coating is 140 μm.

The HA coating on the surface ‘B’ helps in better osteointegration and provide long term stability to the implant 100. The HA coating provided on the metal components facilitates strong adhesion compared to Polyether ether ketone (PEEK) surfaces. Therefore, HA coated surface ‘B’ of the present invention is more stable than a conventional implant with Polyether ether ketone (PEEK) bone contacting surface. A reference is made to the pending patent application number IN202121028444 which discloses a process of coating of hydroxyapatite (HA) on a hip implant.

In an exemplary embodiment, the implant imaging system 200 is described in context of a knee implant 100 as depicted in FIG. 1a. The knee implant 100 may include one or more components including, but not limited to, a femoral component 110, a liner component 130 and a tibial component 150. These components of the knee implant 100 are designed to work together as a functional unit, to replace and provide function of a natural knee joint.

The femoral component 110 may be attached to a femoral head 3 of a knee joint 1 and forms a superior articular surface (not shown). The liner component 130 may form an inferior articulating surface (not shown) with the femoral head 3. The tibial component 150 may include a tibial stem 151 and a tibial base plate 153. The liner component 130 may be coupled to the tibial base plate 153 via any technique including but not limited to press fit mechanism, medical grade adhesives, etc. The tibial stem 151 may be inserted into a marrow cavity of a tibia and the tibial base plate 153 contacts/holds the tibial stem 151.

The knee implant 100 may be made of a polymeric and/or metal material. The polymeric material may include but not limited to poly-ether ether ketone (PEEK), polyethylene, polytetrafluoroethylene (PTFE) or a combination thereof. The metal material may include but not limited to CoCrMo, Ceramics or a combination thereof. All the aforesaid components of the knee implant 100 may be made of different and/or similar material.

In an embodiment, the femoral component 110, the liner component 130 is made of Polyether ether ketone (PEEK) and the tibial component 150 is made of the CoCrMo alloy.

In another embodiment, the knee implant 100 is a metal free implant with all the components made of Polyether ether ketone (PEEK).

In yet another embodiment, the knee implant 100 includes a Polyether ether ketone (PEEK) femoral component 110 which is coupled with an all-polyethylene liner component 130 and tibial component 150 which makes the implant metal free.

Polyether ether ketone (PEEK) may be selected to fabricate the knee implant 100 as it is more pliable as compared to the metal material, therefore may prevent loosening of the knee implant 100 caused by stress shielding and/or bone resorption. PEEK further prevents allergic reactions as well as wear debris which is caused by articulating components. Polyether ether ketone (PEEK) substrate exhibits excellent properties like mechanical toughness, resistance to thermal & chemical degradation and good biocompatibility.

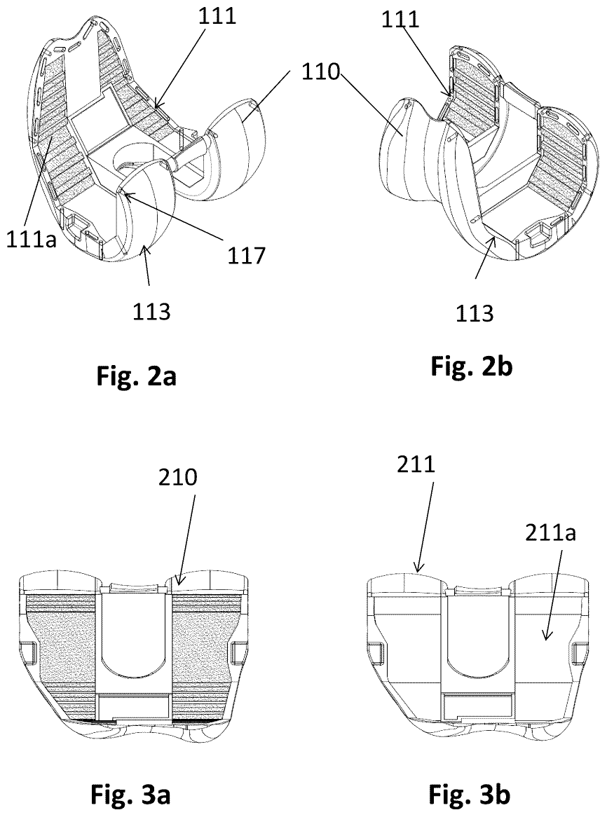

FIG. 2 depicts an exploded view of the femoral component 110 of the knee implant 100. The femoral component 110 may include an inner portion 111 and an outer portion 113. The inner portion 111 may be attached to the femoral head (bone) 3. The outer portion 113 of the femoral component 110 may contact the liner component 130.

The femoral component 110 may be injection molded with Polyether ether ketone (PEEK). Initially when the femoral component 110 is manufactured, it includes smooth surfaces on both side (an inner portion 111 and an outer portion 113). The inner portion 111 and the outer portion 113 of the femoral component 110 may be subjected to a surface modification process. The process of surface modification provides roughness to the inner portion 111 and outer portion 113. The process of surface modification may be performed by means of without limitation grit blasting, sand blasting, micro blasting, air blasting, etc. The process of the surface modification helps to enhance adhesion characteristic by providing rough surface thereby supporting better osseointegration (bone in-growth) and improved implant stability.

The inner portion 111 may have predefined surface roughness which may vary in a range of 5 μm to 100 μm. Surface roughness of the inner portion 111 may extend across at least 50% or at least 70% or at least 80% of the inner portion 111. In an embodiment, the surface roughness of the inner portion 111 is 7 μm which covers around 80% of the inner portion 111.

The femoral component 110 of the knee implant 100 may be provided with the implant imaging system 200. The implant imaging system 200 may provide higher visibility in radiopaque techniques therefore, the femoral component 110 can be easily used with complex geometry bone structure. It further provides strength, radiopacity and better osteointegration to the femoral component 110.

The implant imaging system 200 may be provided on the inner portion 111 of the femoral component 110 of the knee implant 100. The inner portion 111 is chosen as the said portion remains in contact with the native bone where maximum in-bone growth is required. Though, the present invention is described in context of the presence of the implant imaging system 200 on the inner surface, however, the metal imaging system 200 may be disposed on any other surface of the joint implant as well.

The inner portion 111 of the femoral component 110 may include a plurality of slots 111a in order to accommodate the implant imaging system 200. The metal plates 212 of the implant imaging system 200 may be disposed in the slots 111a of the inner portion 111 of the knee implant 100. The plurality of slots 111a may vary as per the implant imaging system 200 to be disposed on the inner portion 111 of the femoral component 110.

The implant imaging system 200 may be in any shape such as without limitation a rectangular plate, a square plate etc. The metal plates 212 of the implant imaging system 200 include a shape complementary to the shape of the slots 111a. The implant imaging system 200 may be disposed in the slots 111a of the inner portion 111 by means of without limitation, snap fit, press fit, adhesives etc. In an embodiment, the implant imaging system 200 is disposed by means of the snap fit mechanism.

In various embodiments, the implant imaging system 200 may be a singular structure and/or multiple structures depending upon the dimension, shape of femoral component 110. In a first exemplary embodiment depicted in FIG. 3, a femoral component 210 having an inner portion 211 is illustrated. The inner portion 211 includes two slots 211a. The two slots 211a may be disposed with implant imaging system 200. In the said embodiment, the implant imaging system 200 includes two metal plates 212 corresponding to the two slots 211a. The present embodiment can be used in patients with complex geometry of the native joint where maximum radiopacity is required.

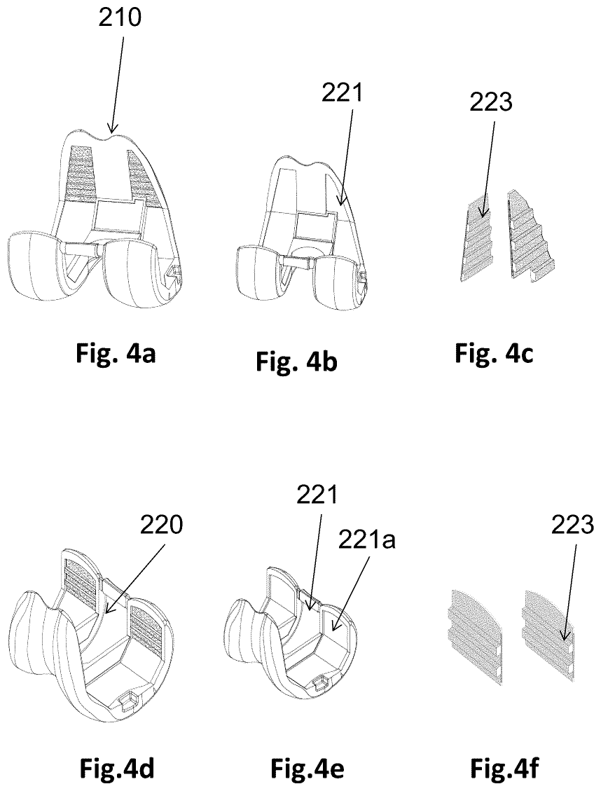

In a second exemplary embodiment, as depicted in FIG. 4, a femoral component 220 includes an inner portion 221 having a plurality of slots 221a. The inner portion 221 may be covered using multiple metal plates 212 of the implant imaging system 200, for example, four metal components 223. The metal plates 212 may be disposed at respective corners of the inner portion 221. The metal plates 212 may be cut as per shape and dimension of the plurality of slots 221a as shown in FIG. 4. The metal components 223 may cover an area ranging from 50% to 80% of the inner portion 221 of the femoral component 210.

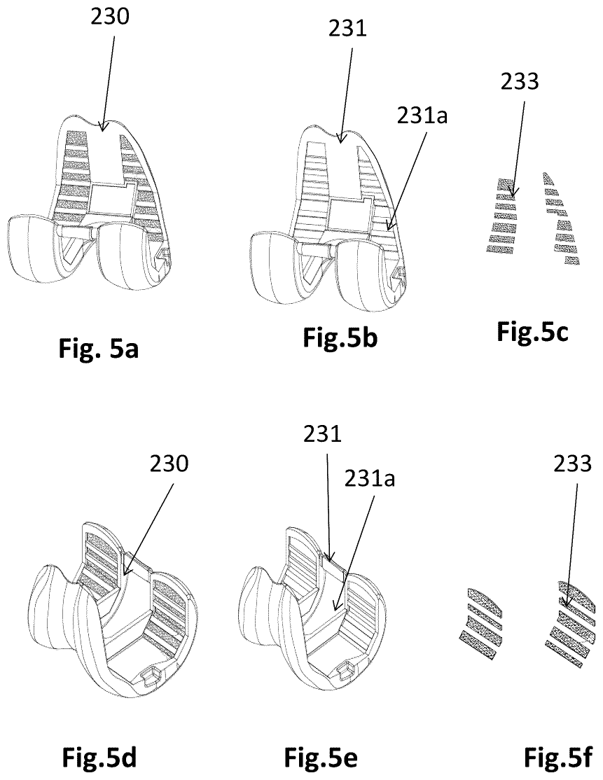

In a third exemplary embodiment, a femoral component 230 with an inner portion 231 is provided as depicted in FIG. 5. The inner portion 231 of the femoral component 230 includes a plurality of slots 231a at a specified distance from each other. A plurality of metal plates 212 may be placed inside the plurality of slots 231a. The plurality of metal components 233 may be shaped and dimensioned as per the plurality of slots 231a. In an exemplary embodiment, the inner portion 231 includes 22 slots and 22 corresponding metal plates 212. The plurality of metal plates 212 may cover an area ranging from 50% to 80% of the inner portion 231 of the femoral component 230. The present embodiment can be used in patients with relatively simple geometry of the native knee joint.

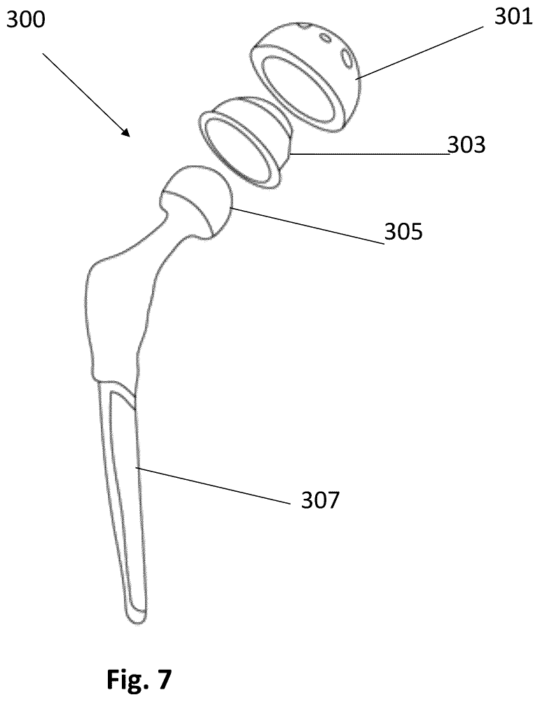

Further, in another exemplary embodiment, the implant imaging system 200 is disclosed in context of a hip implant 300 as depicted in FIG. 7. The hip implant 300 may include an acetabular component 301, a plastic liner 303, a femoral head 305, and a femoral stem 307. In case of the hip implant 300, the metal plate 212 of the implant imaging system 200 is disposed on a top surface of the acetabular component 301. The metal plate 212 may provide adequate visibility to the hip implant 300 for precise placement during implantation and post-surgery follow ups.

Additionally, the knee implant 100 may be provided with a plurality of radiopaque markers. The markers may be provided at predefined positions on the knee implant 100. In an embodiment, markers are provided along the periphery of the femoral component 110 of the knee implant 100.

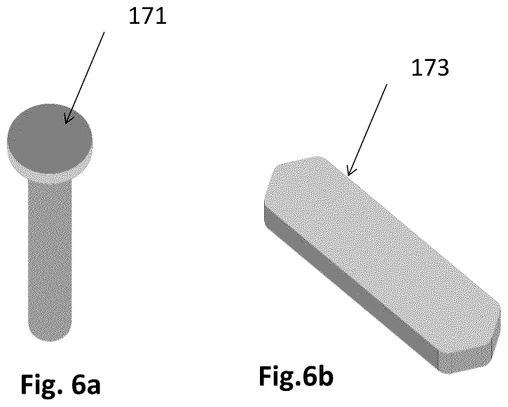

The knee implant 100 may include one or more different type of markers including but not limited to a first marker 171 and a second marker 173 as depicted in FIG. 6a-b. The radiopaque markers may have any shape including but not limited to cylindrical, hexagonal, circular, quadrilateral, cone, hemisphere, ellipsoid, etc. In an embodiment, the first marker 171 is rivet shaped and the second marker 173 is hexagonal shaped. The radiopaque markers may be made from a group of metals including platinum, gold, tungsten, iridium, titanium, tantalum, hafnium or a combination thereof. All of the aforementioned metals have the required biocompatible properties and have a sufficiently large atomic weight so that they are suitable to be used as radiopaque metal.

The femoral component 110 may include a plurality of slots (not shown) and holes 117 depicted in FIG. 2. The holes 117 may be dimensioned to accommodate the first markers 171. The holes 117 may be dimensioned to accommodate the second markers 173. The slots may be disposed at a periphery of the femoral component 110 which is closer to the liner component 130.

Invention is now explained with the help of the following examples.

### Example 1 (Prior Art)

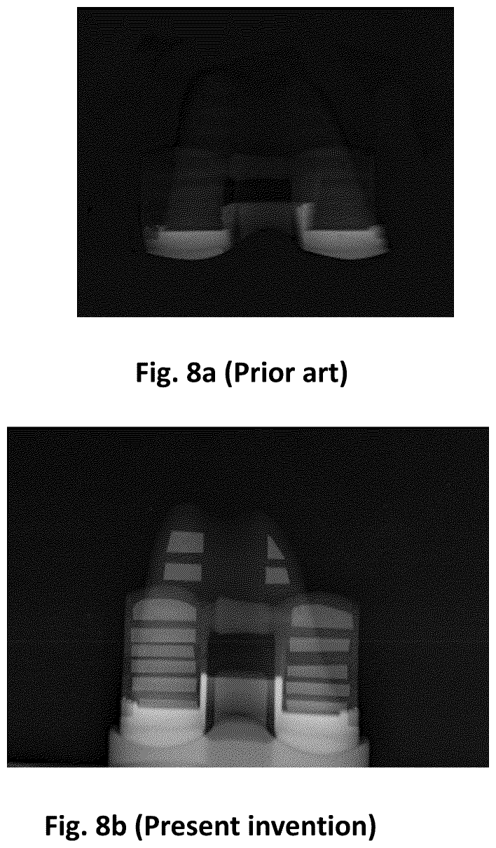

An implant with a femoral component was made from conventional Polyether ether ketone (PEEK) by process of injection moulding. The femoral component was observed under X-ray. The X-ray image as obtained is depicted in FIG. 8a. From the image, it was found that the femoral component was not distinguishable in the X-ray images. Therefore, it was difficult to trace the exact location of the components of the implant in post-surgery assessments.

### Example 2 (Present Invention)

The material and method for the fabrication of the femoral component is similar as mentioned in the example 1. In addition, an implant imaging system was disposed on the inner portion of the femoral component of the knee implant. The implant imaging system included a plurality of metal plates coated with HA material via plasma spray coating technique. The said implant imaging system was attached to the inner portion of the first bone contacting component by a snap fit mechanism.

The implant was tested for visibility under X-ray. The result showed better images as compared to the prior art. The X-ray image of the femoral component of the implant is depicted in FIG. 8b. Further, the HA coated metal plate helped in better osseointegration and thus improved stability of the implant.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. An implant imaging system (200) comprising:
a. one or more metal plates (212) including at least one bone contacting surface ‘B’, the metal plates (212) are disposed on a joint implant; and
b. one or more coatings of osteoconductive material being applied on the bone contacting surface ‘B’ of the metal plates (212) to increase the rate of osteointegration;
wherein the joint implant is made of polymeric material.

2. The implant imaging system (200) as claimed in claim 1 wherein the metal plate (212) is made of one of titanium, tantalum, gold, platinum, hafnium, CoCrMo alloy or a combination (alloy) thereof.

3. The implant imaging system (200) as claimed in claim 1 wherein the metal plate (212) has a thickness ranging from 1 mm to 3 mm.

4. The implant imaging system (200) as claimed in claim 1 wherein the joint implant is one of a knee implant (100), a hip implant (300), a shoulder implant or a spine implant.

5. The implant imaging system (200) as claimed in claim 4 wherein the knee implant (100 includes a femoral component (110) having an inner portion (111).

6. The implant imaging system (200) as claimed in claim 4 wherein the inner portion (111) of the femoral component (110) includes a plurality of slots (111a).

7. The implant imaging system (200) as claimed in claim 6 wherein the metal plates (212) are disposed in the plurality of slots (111a) of inner portion (111) of the knee implant (100).

8. The implant imaging system (200) as claimed in claim 7 wherein the metal plates (212) include a shape complementary to the shape of the slots (111a).

9. The implant imaging system (200) as claimed in claim 5 wherein the knee implant (100) includes a plurality of radiopaque markers.

10. The implant imaging system (200) as claimed in claim 4 wherein the metal plate (212) is disposed on a top surface of an acetabular component (301) of the hip implant (300).

11. The implant imaging system (200) as claimed in claim 1 wherein the osteoconductive material includes hydroxyapatite (HA).

12. The implant imaging system (200) as claimed in claim 11 wherein the hydroxyapatite (HA) coating has a thickness ranging from 110 μm to 190 μm.

13. The implant imaging system (200) as claimed in claim 1 wherein the joint implant is made of Polyether ether ketone (PEEK).
